(19) United States
(12) Patent Application Publication
(10) Pub. No.: US 2008/0234663 A1
(43) Pub. Date: Sep. 25, 2008

(54) Method for Selecting Bolus Doses in a Drug Delivery System

(76) Inventors: Ofer Yodfat, Maccabim-Reut (IL); Gali Shapira, Haifa (IL); Iddo M. Gescheit, Tel-Aviv (IL)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 12/051,400

(22) Filed: Mar. 19, 2008

Related U.S. Application Data
(60) Provisional application No. 60/918,972, filed on Mar. 19, 2007.

(51) Int. Cl. A61M 5/00; G06Q 50/00

(52) U.S. Cl. 604/890.1; 705/2

(57) ABSTRACT

A method, apparatus and system for selecting a bolus dose of a drug in a drug delivery device is disclosed. The method comprises selecting the bolus dose from a pre-determined schedule of bolus doses, wherein each dose corresponds to a range of a body analyte levels.

### RELATED APPLICATIONS

This application claims priority to U.S. Provisional Application Ser. No. 60/918,972 filed Mar. 19, 2007, which is herein incorporated by reference in its entirety.

### FIELD OF INVENTION

Various embodiments described herein relate generally to a field of medical devices. Some implementations relate to a method and device for sustained medical infusion of fluids. Some implementations relate to portable infusion devices and methods for selecting an amount of fluid to be infused by such infusion devices. Some implementations relate to skin securable insulin dispensing devices and methods for selecting an insulin bolus dose.

### BACKGROUND

Diabetes mellitus is a disease of major global importance, increasing in frequency at almost epidemic rates, such that the worldwide prevalence in 2006 is 170 million people and predicted to at least double over the next 10-15 years. Diabetes is characterized by a chronically raised blood glucose concentration (hyperglycemia) due to a relative or absolute lack of the pancreatic hormone-insulin. Within the healthy pancreas, beta cells, located in the islets of Langerhans, continuously produce and secrete insulin according to the blood glucose levels, maintaining near constant glucose levels in the body.

Much of the burden of the disease to the patient and to health care resources is due to the long-term tissue complications, which affect both the small blood vessels (microangiopathy, causing eye, kidney and nerve damage) and the large blood vessels (causing accelerated atherosclerosis, with increased rates of coronary heart disease, peripheral vascular disease and stroke). The Diabetes Control and Complications Trial (DCCT) demonstrated that development and progression of the chronic complications of diabetes are greatly related to the degree of altered glycemia as quantified by determinations of glycohemoglobin (HbA1c). [DCCT Trial, N Engl J Med 1993; 329: 977-986, UKPDS Trial, Lancet 1998; 352: 837-853. BMJ 1998; 317, (7160): 703-13 and the EDIC Trial, N Engl J Med 2005; 353, (25): 2643-53]. Thus, maintaining euglycemia by frequent glucose measurements and adjustment of insulin delivery accordingly is of utmost importance.

Insulin pumps can deliver rapid acting insulin 24 hours a day through a catheter placed under the skin. The total daily insulin dose can be divided into basal and bolus doses. Basal insulin can be delivered continuously over 24 hours, and can keep the blood glucose levels in range between meals and overnight. Diurnal basal rates can be pre-programmed or manually changed according to various daily activities. Insulin boluses can be delivered before meals or during episodes of high blood sugar levels to counteract carbohydrates loads.

The amount of insulin in the administered bolus depends on several parameters:
Amount of carbohydrates (Carbs) to be consumed, alternatively defined as “servings”, wherein 1 serving=15 grams of Carbs.
Carbohydrate-to-insulin ratio (CIR), i.e. the amount of carbohydrates balanced by one unit of insulin.
Insulin sensitivity (IS), i.e. the amount of blood glucose value lowered by one unit of insulin.
Current blood glucose level (BSC)
Target blood glucose level (BST), i.e. the desired blood glucose level. BST for most people suffering from diabetes is in the range of 90-130 mg/dL before a meal, and less than 180 mg/dL 1-2 hours after the start of a meal.
Residual insulin, i.e. the amount of stored insulin remained in the body after recent bolus delivery that is still active. This parameter is relevant when there is a short time interval between consecutive boluses (i.e. less than 5 hours).

Insulin pump users regularly calculate, or make educated estimations of appropriate pre-meal insulin bolus doses. These calculations or estimations can be based on the above mentioned parameters, to effectively control the blood glucose levels and maintain euglycemia. However, patients may miscalculate or make inadequate estimations that can lead to under or overdosing of insulin, resulting in hypo or hyperglycemia accordingly. Inadequate estimations can be, for example, due to misevaluation of the carbohydrate content in the intake.

There are known in the art portable insulin pumps provided with bolus calculating means based on patient inputs of meal carbohydrate content and glucose levels. For example, in U.S. Pat. No. 6,936,029 assigned to Medtronic MiniMed, such a pump provided with a bolus calculator and an algorithm for calculating the amount of insulin to be administered is described. The algorithm is based on a formula for calculating a bolus, depending on the user's IS, CIR, target BG and user inputs of blood glucose (BG) and carbs intake.

If the current BG is higher than the target BG, the recommended bolus is calculated as:

$$Recommended bolus = \underbrace{( TC / CIR )}_{“ Food estimate ”} + {\underbrace{( BSC - BST )} /}_{“ Correction estimate ”} IS - RI$$

Wherein TC=total amount of carbohydrates; CIR=carbohydrate-to-insulin ratio; BST=target blood sugar; BSC=current blood sugar; IS=Insulin sensitivity; RI=remaining insulin (i.e. “residual insulin”).
If the current BG is lower than the target BG, the recommended bolus is calculated as:

Recommended bolus=(TC/CIR)+(BSC−BST)/IS

If the current BG is higher than the low target BG and lower than the high target BG (e.g. current blood (BSC) glucose=105 mg/dL, target range (BST)=90-130 mg/dL) then the recommended bolus is calculated as:

Recommended bolus=(TC/CIR)+0

The residual insulin should be subtracted only from the “correction estimate” of the formula (not from the food estimate).

For a current BG that is above the high target, if the residual insulin is more than the “correction estimate”, the formula's “correction portion” becomes zero (0). For a current BG that is below the low target, if the active insulin is more than the correction estimate, the active insulin is not considered. Thus, parameters input should initially be entered by the user and then the bolus calculation takes place.

Several drawbacks are associated with the approach of user's data input followed by bolus calculation. These drawbacks are:
The need for data inputs complicates bolus calculator user interface because it dictates navigation among several non-user friendly consecutive displays.
Young children may find it difficult to master the bolus calculator's interface since it requires reading and typing alpha-numeric parameters.
Users, especially young children, may find it difficult to estimate the exact carbs load.
Data inputs and subsequent calculations according to inputs are prone to errors. For example, the user may erroneously insert a carb load of 100 g instead of 10 g. Such an error may be associated with serious insulin overdose resulting in a life threatening hypoglycemia.

Application US2005/0065760 assigned to Insulet Ltd. describes even a more complicated algorithm for calculating and suggesting doses of insulin to the user. A range of suggested doses is provided to the user wherein the mid-range value is calculated based on an algorithm, which takes into consideration the desired (target) BG and current BG. A correction factor is added to and subtracted from the mid-range value to yield a range of recommended doses. The correction factor takes into account the error of the glucometer, contemplated physical exercise, age of the blood sample etc. The patient chooses a bolus dose from within the recommended range, which has been obtained by the calculation. It is apparent that implementation of this sophisticated algorithm, which requires inputting of data with subsequent calculation would be associated with the same disadvantages as mentioned above.

There are more known methods for calculating bolus doses, like, for example, the method described in U.S. Pat. No. 6,691,043 assigned to Maxi-Med Ltd. This method takes into consideration the user's carbohydrate ratio determined as a function of time and then the corrected ratio is used for the calculation of the recommended dose, according to the following equation:

Bolus=(C/L)+(BSR−BST)/IS

Wherein C=total amount of carbohydrates,
L=corrected carbohydrate ratio, BST=target blood sugar, BSR=blood sugar reading,
IS=Insulin sensitivity.

Besides the above-mentioned disadvantages, the known methods based on data inputs with subsequent calculation suffer from an intrinsic disadvantage-relevant factors that should be taken into consideration can be overlooked. Among these factors are the anticipated and/or previous exercise levels and duration, fat and protein contained in the intake, variation of parameters during time of day (e.g. the CIR is not constant throughout the day), and inter-individual and intra-individual absorption rate variability (relevant for the residual insulin parameter). Furthermore the inaccuracy of the current BG measurement due to the error of the glucometer (up to 20% error) is not reflected in the abovementioned equations. In addition, the estimation of the carbohydrate content in the intake made by the user is inevitably imprecise.

All these reasons render the apparent accurateness of the input based calculated bolus questionable, and its apparent high resolution possibly misleading. It can be stated that the promised high resolution of the calculated insulin doses provided by the prior art bolus calculators is of minimal clinical significance.

### SUMMARY OF THE INVENTION

Techniques and devices are described for providing a method and a system for selection of a bolus dose of a drug in a drug delivery device. Some aspects provide a method for selecting a bolus dose of a drug in a drug delivery device comprising selecting the bolus dose from a pre-determined schedule of bolus doses, wherein each dose corresponds to a range of a body analyte levels. In one variation, the pre-determined schedule of doses corresponds to a bolus-grid. In another variation the drug comprises insulin and the range of the body analyte levels corresponds to a range of blood glucose levels. In another variation, the range of the blood glucose levels is represented as a qualitative descriptive parameter (QDP). In yet another variation, the QDP comprises a plurality of terms, each associated with a pre-determined blood glucose level or a pre-determined range of blood glucose levels. In a further variation, the QDP terms are selected from the group consisting of high, normal and low. The bolus doses can, for example, be determined by a plurality of parameters. The parameters can be selected from the group consisting of time elapsed from a last meal, an amount of one or more previous bolus doses, desired/target blood glucose level (TBG), carbohydrate-to-insulin ratio (CIR) and insulin sensitivity (IS).

Another aspect provides a method for selection of a bolus dose of a drug in a drug delivery device comprising selecting the bolus dose from a pre-determined schedule of bolus doses, wherein each dose corresponds to a range of a nutritional consumable. In one variation, the pre-determined schedule of doses corresponds to a bolus-grid. In another variation, the drug is insulin and the nutritional consumable is at least one of carbohydrate, fat and protein. The range of the nutritional consumable, in one example, can be represented as a qualitative descriptive parameter (QDP). The QDP can comprise a plurality of terms, each associated with a pre-determined range of the nutritional consumable. The terms can be selected from the group consisting of small, medium and large. The bolus doses can be determined by plurality of parameters. The parameters can be selected from the group consisting of time elapsed from a last meal, an amount of one or more previous bolus doses, desired/target blood glucose level (TBG), carbohydrate-to-insulin ratio (CIR) and insulin sensitivity (IS). These parameters can also be selected from the group consisting of intensity of physical activity, duration of physical activity, menstrual cycle, concomitant administration of other drugs, the drug injection site, glycemic index, stress and fever.

Another aspect provides a method for selection of a bolus dose of a drug in a drug delivery device comprising selecting the bolus dose from a pre-determined schedule of bolus doses, wherein each bolus dose corresponds to one or more glucose levels and one or more nutritional consumables. In one variation, each bolus dose can correspond to a range of glucose levels and a range of nutritional consumables. The schedule of doses can correspond to a bolus-grid.

In yet another aspect, an apparatus for selecting a bolus dose is provided. In one implementation the apparatus comprises means for providing a pre-determined schedule of bolus doses, wherein each dose corresponds to a range of body analyte levels; and means for selecting a bolus dose from the pre-determined schedule of bolus doses. In one variation, the apparatus can further comprise a remote control unit for controlling the means for selecting the bolus dose. The means for providing the pre-determined schedule of bolus doses can comprise a display for displaying the pre-determined schedule of bolus doses. The means for providing the pre-determined schedule of bolus doses can further include means for indicating a bolus dose based on averaged values of previously used blood glucose levels and a carbohydrate intakes during a time interval. The means for providing the pre-determined schedule of bolus doses can further include means for presenting a subset of the predetermined schedule of bolus doses which corresponds to an averaged value of previously used blood glucose levels during a time interval. The means for selecting the bolus dose can also comprise either a touch sensitive screen which operates in conjunction with the display or one or more selection buttons/switches. In a further variation, the apparatus comprises a pump for dispensing bolus doses into a user. In yet another variation, the apparatus further comprises a body analyte level monitor for monitoring a body analyte level of a user; and, a processor for matching the body analyte level of the user to the range of body analyte levels.

In some aspects, an apparatus for selecting an insulin bolus dose is provided. The apparatus can comprise means for providing a pre-determined schedule of bolus doses as a function of one or more inputs, said inputs comprising at least one of a carbohydrate-to-insulin ratio (CIR), insulin sensitivity (IS) value and a body analyte level of a user; means for adjusting the schedule of bolus doses based on the inputs; means for selecting a bolus dose from the pre-determined adjusted schedule of bolus doses. The means for providing the pre-determined schedule of bolus doses can comprise a display for displaying the pre-determined schedule of bolus doses. The means for providing the pre-determined schedule of bolus doses can further include means for indicating a bolus dose based on averaged values of previously used blood glucose levels and a carbohydrate intakes during a time interval. The means for providing the pre-determined schedule of bolus doses can further include the means for presenting a subset of the predetermined schedule of bolus doses which corresponds to an averaged value of previously used blood glucose levels during a time interval. Finally, the means for selecting the bolus doses can comprise either a touch sensitive screen which operates in conjunction with the display or one or more selection buttons/switches. The apparatus for selecting an insulin bolus dose can also comprise a dispensing unit for dispensing bolus doses into the user. It can also comprise at least one monitor for monitoring the body analyte level of the user.

In one implementation, the means for adjusting the schedule of bolus doses can comprise selecting means for selecting at least one of the CIR and IS values from a schedule. The means for adjusting the schedule of bolus doses can comprise means for selecting rules for determining the CIR and IS values. In some implementations, the apparatus can further comprising a display configured to display values corresponding to the inputs in a form of a list, a table or a graphical indication.

In another aspect, a method for selecting an insulin bolus dose is provided. In one implementation, this method comprises receiving at least one value selected from the group consisting of a carbohydrate-to-insulin ratio (CIR), an insulin sensitivity (IS) value and a target blood glucose (TBG) level of a user; retrieving a pre-determined schedule of bolus doses based on the at least one determined value; presenting the pre-determined schedule of bolus doses for selection to the user; selecting at least one bolus dose from the pre-determined schedule of bolus doses based on the user's blood glucose level and the user's nutritional consumable load.

In one implementation, the carbohydrate-to-insulin ratio (CIR) and the insulin sensitivity (IS) values are selected from a schedule by the user. The schedule can be determined by using rules for estimation of the carbohydrate-to-insulin ratio (CIR) and the insulin sensitivity (IS) values. The estimation of the carbohydrate-to-insulin ratio (CIR) and the insulin sensitivity (IS) values, for example, can be based on averaged values of total daily doses (TDD).

In yet a further aspect, a system for drug dispensing is provided. In one implementation, this system comprise a remote control unit with a display for providing a pre-determined schedule of bolus doses, wherein each dose corresponds to a range of nutritional consumable load and to a range of body analyte levels; a user interface for selecting a bolus dose from the pre-determined schedule of bolus doses; a dispensing unit for dispensing the bolus dose into a user, wherein the dispensing unit receives instructions for dispensing the bolus dose from the remote control unit. The dispensing unit can comprise a reusable part which includes a processor and at least a portion of a driving mechanism and a disposable part which includes a reservoir. The system can also comprise a cradle unit. In some implementations, the system can comprise a dispensing unit for delivering fluid into the user's body and a suitable sensing means for sensing, measuring and monitoring an analyte concentration level in the user's body. For example, the fluid can be insulin and the analyte can be glucose. In some implementations, the dispensing unit and the sensing means operate as a semi-closed loop system. Some embodiments provide a device that can employ a simplified, easy to use method allowing selection of appropriate insulin bolus from a plurality of pre-determined insulin boluses. The feature implementing the method for determining the current insulin bolus will be referred-to further as—a “bolus selector”. The bolus selector can be implemented in a stand alone device, or it can be implemented in a glucometer, an infusion pump, a delivery pen, a PC or any other device which can be used by the diabetes patient. Some embodiments can provide a device that can monitor glucose concentration levels and implement an easy to use method for selecting a recommended insulin bolus from a pre-determined list of boluses. Some embodiments can provide a device that can dispense insulin and apply a simplified easy to use method for selecting an insulin bolus.

Some embodiments can provide a device that can monitor glucose concentration levels and dispense insulin according to a bolus, which can be selected by a simplified, easy to use bolus selecting method. Some embodiments provide a device that can continuously monitor body glucose levels and employ a simplified, easy to use method for selecting an insulin bolus.

Some embodiments provide a device that can continuously monitor body glucose levels and can concomitantly deliver insulin bolus into the body selected by virtue of a simplified, easy to use bolus selecting method. Some embodiments provide a device, which can be miniature, discreet, economical for the users and highly cost effective for the payer and which can employ a simplified, easy to use method to select an insulin bolus.

Some embodiments provide a system comprising a miniature skin securable patch that can continuously dispense insulin, according to a bolus which is selected by a simplified, easy to use method. Some embodiments provide a device that can includes a skin securable dispensing patch unit composed of two parts. The dispensing patch unit may be attached to the skin directly, or by virtue of a needle unit.

Some embodiments provide a device provided with a dispensing patch unit that can be disconnected and reconnected. Some embodiments provide a system comprising a miniature skin securable patch that can continuously dispense insulin and monitor body glucose concentration levels, wherein this system employs a simplified, easy to use method to select an insulin bolus.

Some embodiments provide a semi-closed loop system that can monitor glucose levels and dispense insulin according to said sensed glucose levels and according to a bolus selected by a simple method. This method can be implemented in a miniature single device, discreet, economical for the users and highly cost effective for the payer.

Some embodiments provide a device that contains an insulin infusion patch unit comprising a disposable part and a reusable part. The reusable part can contain all relatively expensive components and the disposable part can contain cheap components, thus providing a low cost product for the user and a highly profitable product for the manufacturer and the purchaser. The device can employ a simplified, easy to use method for selecting a bolus. Some embodiments provide a device that comprises an insulin infusion patch unit that can be remotely controlled. Some embodiments provide a simple, convenient, easy to use method that can help the pump user select and deliver a desired bolus dose.

Some embodiments provide a simple, convenient, easy to use method that can help the pump user to select an appropriate bolus out of pre-determined dosage selections. This method avoids data input errors and subsequent miss-calculation according to algorithms' related inputs. In selecting a recommended bolus from pre-determined choices there is no data input and consequently no input related errors.

Some embodiments provide a dispensing device which delivers a selected bolus dose and can receive glucose levels readings. Some embodiments provide a dispensing device that monitors glucose concentration levels and dispenses insulin boluses according to a selection method. Some embodiments incorporate the bolus selection method in a device that continuously monitors body glucose levels.

Some embodiments incorporate the bolus selection method in a device that continuously monitors glucose levels and can concomitantly deliver insulin into the body. Some embodiments incorporate the bolus selection method in a device, which is miniature, discreet, economical for the users and highly cost effective for the payer.

Some embodiments incorporate the bolus selection method in a device configured as a miniature patch that can be secured to the skin and can continuously dispense insulin. Some embodiments incorporate the bolus selection method in a device that comprises a dispensing patch unit that can be disconnected from and reconnected to the patient, thereby allowing temporary removal in cases such as hot bath, sauna, intimacy, etc. Disconnections and reconnections should neither harm various components of the patch, like the pumping mechanism, the needle, nor the surrounding tissue and/or the patient.

Some embodiments incorporate the bolus selection method in a device that contains a dispensing patch unit and a needle unit, and that the needle unit is securable to the skin, and where the dispensing patch unit can be connected to and disconnected from the needle unit upon patient discretion. Some embodiments incorporate the bolus selection method in a device that comprises an insulin infusion patch unit that can be remotely controlled.

Some embodiments implement the bolus selection method in a remote control unit of an insulin dispensing device. Some embodiments incorporate bolus selection method in a device comprising a miniature skin securable patch that can continuously dispense insulin and monitor body glucose concentration levels.

Some embodiments provide a device that contains a semi-closed loop system that can monitor glucose levels and dispenses insulin according to said sensed glucose levels and the bolus selection method. Some embodiments provide a device that contains a semi-closed loop system, which is miniature, discreet, economical for the users and highly cost effective for the payer. Some embodiments provide a method for monitoring the CIR and IS value and provide the diabetes patient with a better clinical follow up.

### BRIEF DESCRIPTION OF THE DRAWINGS

FIG. 1 illustrates one variation of the insulin infusion device comprising an insulin dispensing unit and a remote control unit that contains a bolus selector.

FIG. 2 shows a block diagram representing one example of a method for selecting an insulin bolus dosage.

FIG. 3 shows a block diagram representing another example of a method for selecting an insulin bolus dose.

FIGS. 4a-4c show three bolus grids with recommended insulin doses based on meal carbohydrate content and current blood glucose measurements.

FIG. 5 shows an embodiment of the bolus selector user's interface.

FIG. 6 shows another embodiment of the bolus selector user's interface.

FIG. 7 shows another embodiment of the bolus selector user's interface.

FIG. 8 shows another embodiment of the bolus selector user's interface where the carb intake is used for bolus selection.

FIG. 9 shows another embodiment of the bolus selector user's interface.

FIGS. 10a-10f show some implementations of the bolus selector user's interface.

FIG. 11 is a schematic drawing of an insulin infusion device including a skin securable dispensing unit composed of a reusable part and a disposable part, and a remote control unit that contains the bolus selector.

FIGS. 12a-12c show some implementations of an insulin infusion device containing blood glucose monitor for providing blood glucose (BG) readings to the bolus selector.

FIGS. 13a-13b show some implementations of an insulin infusion device containing continuous subcutaneous glucose monitor providing blood glucose readings (BG) to the bolus selector.

FIGS. 14a-14b show some implementations of the bolus selector.

FIG. 15 provides a block diagram representing some implementations of data acquisition modes.

FIG. 16 shows some implementations of the bolus selector located in the remote control unit and PC.

FIGS. 17a-17d show different grids that can be incorporated in some implementations of the bolus selector.

FIGS. 18a-18c provide several examples of grids with their corresponding minimal undershoot and maximal overshoot BG values.

### DETAILED DESCRIPTION OF THE INVENTION

A method and a device for selecting a bolus dose of a drug is provided. In one implementation the method comprises selecting the pre-determined bolus dose from a pre-determined schedule of bolus doses. The pre-determined schedule of bolus doses can provide one or more predetermined doses corresponding to a range of a body analyte levels.

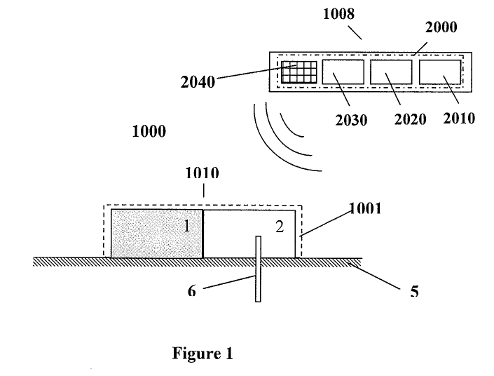

For example, FIG. 1 shows an insulin infusion device comprising a dispensing unit (1010) configured as a patch that can be secured to the user's skin (5). The device can also comprise a remote control unit (1008) that can communicate with the dispensing patch unit (1010), in some implementations, for programming, user inputs and data acquisition purposes.

Manual inputs can be carried out, for example, by one or more buttons located on the dispensing patch unit (1010). The dispensing patch unit (1010) can be composed of one housing (1001) or two housings comprising reusable (1) and disposable (2) parts as shown in our previous patent application U.S. Ser. No. 11/397,115 (herein incorporated by reference in its entirety). The dispensing patch unit (1010) can also comprise a cannula (6) that can penetrate the skin (5) to allow delivery of insulin.

The dispensing patch unit (1010) can be directly attached to the user's skin (5) by adhesives (not shown) or can be attached to a dedicated needle unit (not shown) that can be adhered to the patient skin (5) and can allow connection and disconnection of the dispensing patch unit (1010).

In some implementations, the remote control unit (1008) can contain the bolus selector (2000), processor (2010), memory (2020), input means (2030) display (2040) and other indication means (not shown) such as audible and vibrational means. The input means (2030) are preferably provided for the bolus selector (2000) and for dispensing patch unit (1010) programming.

Options for selection of insulin bolus can be presented by the bolus selector (2000) on the display (2040) as a table with cells containing pre-determined bolus dosages. The appropriate cell with corresponding insulin dose can be selected from a pre-determined table according to, for example, at least one of the parameters consisting of blood glucose, carb content, and previous meal (insulin residue). Such a table with pre-determined bolus dosages will be referred-to as a “bolus-grid”.

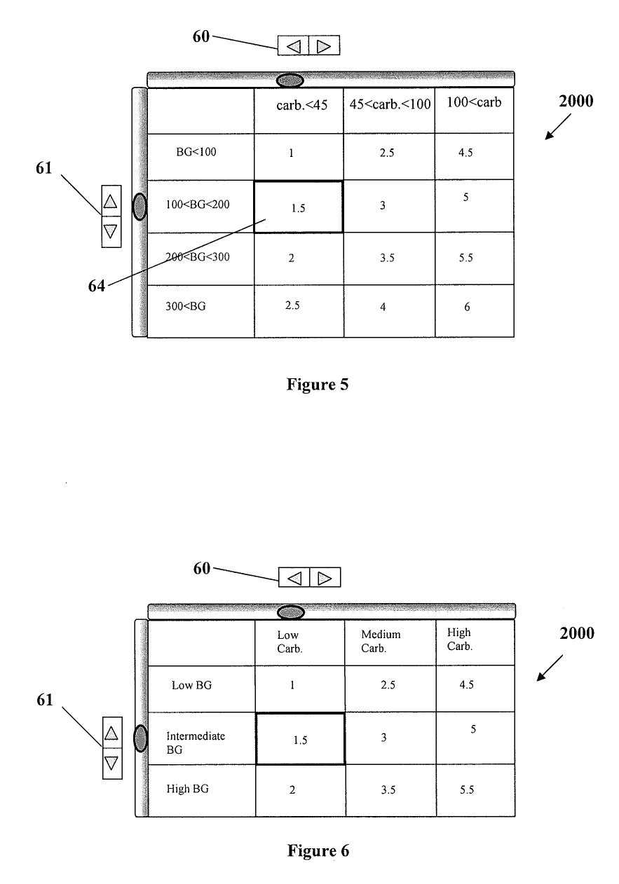

The bolus-grid cells can comprise pre-determined bolus doses corresponding to possible combinations of the current blood glucose level and the user's approximation of the carb load in the related meal (examples of such grids are shown in FIGS. 5-8). The bolus-grids can be stored in the memory which can be provided in the bolus selector (2000) itself or in any other memory such as the memory located in the remote control unit.

The bolus selector (2000) can contain many bolus grids. The values of the bolus dosages in each bolus-grid can be pre-determined according to BG and carb intake. The user's specific bolus-grid can be retrieved from a set of bolus grids stored in the bolus selector's (2000) memory. Each set can correspond to a specific combination of target BG, IS value, CIR, and insulin residue. Each grid in the set can also correspond to a different “residual insulin” value.

In one implementation, the method(s) for selecting a suitable insulin bolus to be administered can be based on data that takes into account at least one of the following parameters: current blood glucose levels and carbohydrate intake. The method(s) can also be based on selection of the optimal dose from pre-determined values of the insulin bolus.

In one implementation, the user can select the insulin bolus from a pre-determined schedule of bolus doses, corresponding to a ranged numerical blood glucose (BG) values (e.g. BG<100, 100<BG<200, 200<BG<300, 300<BG). Each blood glucose range can correspond to a pre-determined bolus dose (e.g. 100<BG<140=4 units, 140<BG<180=6 units, etc.) to be selected by the user.

Similarly, the user can select the insulin bolus dose from pre-determined values, which correspond to a carbohydrate (“carb” or “carbs”) load presented by a ranged numerical carbohydrate values (e.g. carbs<45, 45<carbs<105, carbs <105), rather than discrete values. In some implementations, the carbs load may be expressed in “servings” instead of grams. For example, one serving of a carbohydrate food can contain 15 grams of carbohydrate. In some implementations, the carbs can be presented in ranges of servings (e.g. serving <3, 3<servings<7, serving>7).

The blood glucose level can be presented as a qualitative descriptive parameter (QDP) (e.g. high, normal, or low BG). Similarly, the carbohydrate intake can also be presented as a qualitative descriptive parameter (QDP) (e.g. snack, small meal, medium sized meal, large meal). This qualitative parameter can be nominated according to approximate correspondence between the size of the meal and its carbohydrate load. Presenting carbohydrate intake as a qualitative descriptive parameter instead of a quantitative parameter can be especially convenient for young children.

For example, the carbohydrate intake and the current blood glucose level can be designated as qualitative parameters as illustrated by the non-limiting Table 1 below. In some implementations, the table can be configured as a grid, a lookup table or database. This grid can be stored and retrieved from the memory of the bolus selector. The carbs intake can be presented in the upper row of the grid, while blood glucose levels (BG) can be presented in the left column of the grid. In some implementations, each one of the carbs intake and the BG parameters can be presented as a qualitative descriptive parameter (QDP).

In one implementation, the table can represent a matrix of cells containing values of recommended insulin boluses, which have been pre-determined for each combination of the carbs intake and BG. These values can be stored as a readily available database in the memory of the bolus selector. The pre-determined database can be displayed by the bolus selector graphically as plurality of cells arranged in a grid, such as a “bolus-grid.” The bolus grid can be displayed by the bolus selector. The bolus-grid can allow the user to select a cell from within the grid depending on a combination of a BG and a carbohydrate intake range values.

TABLE 1
Example of a bolus-grid

| | | Carbs Intake | | |
|---|---|---|---|---|
| | | Small | Medium | Large |
| BG | Low | 1.5 U | 2.5 U | 3 U |
| | Normal | 2.5 U | 3.5 U | 4 U |
| | High | 3 U | 4 U | 5.5 U |

In one implementation, the bolus selector can enable the user to input the numerical value of blood glucose instead of a qualitative descriptive parameter (QDP). The BG numerical value can then be presented in the bolus-grid as a suitable qualitative parameter, and then only a row of the bolus-grid that corresponds to the relevant BG range can be displayed to the user instead of the whole bolus-grid. For example:

Measured BG=225→Pre-determined bolus-grid row-High

Presented row:

| | Small | Medium | Large |
|---|---|---|---|
| High | 5 U | 7 U | 9 U |

In one implementation, the pre-determined insulin bolus doses that correspond to a selected current BG range and carbs intake range (hereinafter known as “corresponding doses”) can be pre-determined according to a formula, which can take into consideration the user's CIR and IS. By virtue of this provision, the bolus-grid doses can be adjusted to the user's individual insulin needs.

The adjusted insulin doses can then be stored in the memory of the bolus selector as a plurality of bolus-grids. For example, each stored bolus-grid can correspond to a certain combination of CIR and IS values.

In one implementation, the user can set the CIR and IS values during the initial setup of the bolus selector. The user can also provide a “rule” from which the CIR and IS values can be obtained (as illustrated in Tables 2 and 3 shown below). For example, high and low CIR and IS values correspond to smaller or higher bolus doses respectively.

In some implementations, additional parameters, such as target blood glucose levels, can also be used. In some implementations, grid selection can be carried out according to the IS value, CIR, and target blood glucose levels which can vary throughout the day.

In one implementation, the bolus-grids can be switched by the user in order to comply with possible and/or predicted changes in CIR. That is, the database with pre-determined bolus doses may be stored as a plurality of sets of bolus-grids and the user may choose the suitable bolus-grid sets according to a predicted and established variability in CIR. This, for example, can be particularly useful for adolescent users due to the relatively frequent changes in CIR during puberty.

In another implementation, additional parameters, such as time lapsed from last meal and amount of previous bolus dose can also be considered according to pre-determined, known in the art, insulin absorption rate tables. Each current bolus-grid can be corresponded to residual insulin time/dose grids. For example, the residual insulin can be extracted from a chart, as illustrated in Table 4. In some implementations, the determined amount of residual insulin can be displayed by the bolus selector to inform the user.

In one implementation, one or more last days' boluses (time and dose) can be stored in the bolus selector in memory. Accordingly, average total daily dose or average dose during a specific time interval (e.g. average boluses administered in the last week between 6 to 10 a.m.) can be determined and/or presented. Thus, the bolus selector can indicate a bolus dose even prior to loading the BG and carbs for retrieval of a pre-determined bolus-grid and recommendation of a specific dose.

The indicated bolus dose can be presented in the corresponding bolus-grid as a first preferable choice. For example, this feature can be especially beneficial for users with routine daily intakes or for patients who don't measure BG before every meal.

In one embodiment, the bolus doses can be pre-determined by averaging the values corresponding to the same time interval in the same basal profile. For example, only the boluses given between 6 to 10 a.m. in the last 7 “weekend” profiles will be averaged together.

In one implementation, the averaged bolus will be presented to the user only if the standard deviation is small. If a significant deviation is noted, the averaged bolus value is not presented. In another implementation, the bolus selector can alert the user if the selected bolus is different from the average by, for example, a certain amount (e.g., a considerable amount).

In another implementation, the total daily bolus dose average can be used for insulin sensitivity (IS) and carb to insulin ratio (CIR) bolus selector calibration. In some embodiments, the bolus selector can alert the user of the change in insulin sensitivity and/or carb to insulin ratio, which can be crucial for clinical follow-up and treatment adjustments.

In some implementations, the preliminary insulin sensitivity (IS) can be determined according to the “2200 to 1600 rules” commonly used by type one diabetes patients using rapid acting insulin (e.g. Humalog, Novolog). Such rules are incorporated herein by reference in their entirety. The user IS can be determined by dividing the value corresponding to an appropriate rule by the total daily dose of rapid-acting insulin (e.g. if the total daily insulin dose is 40 units and the 1800 rule is used, the insulin sensitivity factor would be 1800 divided by 40=45 mg/dl/unit). For example, Table 2 shows the point drop per unit of insulin (insulin sensitivity) according to the various rules (adapted from Using Insulin© 2003).

TABLE 2
Insulin sensitivity table grid, point drop per unit of insulin

| Total daily insulin | 2200 Rule | 2000 Rule | 1800 Rule | 1600 Rule |
|---|---|---|---|---|
| dose (TDD) [IU/day] | [mg\dL] | [mg\dL] | [mg\dL] | [mg\dL] |
| 20 | 110 | 100 | 90 | 80 |
| 25 | 88 | 80 | 72 | 64 |
| 30 | 73 | 67 | 60 | 53 |
| 35 | 63 | 57 | 51 | 46 |
| 40 | 55 | 50 | 45 | 40 |
| 50 | 44 | 40 | 36 | 32 |
| 60 | 37 | 33 | 30 | 27 |
| 75 | 29 | 27 | 24 | 21 |
| 100 | 22 | 20 | 18 | 16 |

The carb to insulin ratio (CIR) can be determined, for example, according to the “450 to 500 rules” commonly used by type one diabetes patients using rapid acting insulin (e.g. Humalog, Novolog). The user CIR can be determined by dividing the value corresponding to appropriate rule by the total daily dose of rapid-acting insulin (e.g. if the total daily insulin dose is 40 units and the 450 rule is used, the carb to insulin ratio (CIR) would be 450 divided by 40=11 gram).

Table 3 shows carbs (in grams) covered by 1 unit of insulin (CIR ratio) according to the various rules (adapted from Using Insulin© 2003).

TABLE 3
Rules for the determination of CIR

| Total daily insulin dose | | |
|---|---|---|
| (TDD) [IU/day] | 500 Rule [gram] | 450 Rule [gram] |
| 20 | 25 | 23 |
| 25 | 20 | 18 |
| 30 | 17 | 15 |
| 35 | 14 | 13 |
| 40 | 13 | 11 |
| 50 | 10 | 9 |
| 60 | 8 | 8 |

The residual insulin can be determined according to the pharmacokinetics of rapid acting insulin (e.g. Humalog, Novolog).

Table 4 shows the units of residual insulin after 1-5 hours post a previous given bolus (adapted Using Insulin© 2003).

TABLE 4
Insulin residue, insulin units left to work

| Dose | | | | | |
|---|---|---|---|---|---|
| Given | Units left to work after: | | | | |
| [IU] | 1 Hr | 2 Hr | 3 Hr | 4 Hr | 5 Hr |
| 1 | 0.8 | 0.6 | 0.4 | 0.2 | 0 |
| 2 | 1.6 | 1.2 | 0.8 | 0.4 | 0 |
| 3 | 2.4 | 1.8 | 1.2 | 0.6 | 0 |
| 4 | 3.2 | 2.4 | 1.6 | 0.8 | 0 |
| 5 | 4.0 | 3.0 | 2.0 | 1.0 | 0 |
| 6 | 4.8 | 3.6 | 2.4 | 1.2 | 0 |
| 7 | 5.6 | 4.2 | 2.8 | 1.4 | 0 |
| 8 | 6.4 | 4.8 | 3.2 | 1.6 | 0 |
| 9 | 7.2 | 5.4 | 3.6 | 1.8 | 0 |
| 10 | 8.0 | 6.0 | 4.0 | 2.0 | 0 |

In one implementation, the recommended bolus dose can be selected by the user from a displayed graph wherein one axis indicates ranges of current BG levels and another axis indicates ranges of carbohydrate content. In another implementation, the recommended bolus dose can be automatically selected by the bolus selector device upon inputs of at least a range of current BG levels and a range of carbs content. In yet another implementation, the user can accept automatically selected recommended dose and deliver the bolus accordingly.

In another embodiment, the automatically selected dose can be delivered without a user interface notification. For example, the user can be notified prior to bolus administration and can suspend the automatic delivery or select an alternative dose for delivery.

In one implementation, the bolus selection method can be implemented in an insulin infusion device comprising insulin dispensing patch unit and a remote control unit, wherein a glucose sensing means (e.g. glucometer) is integrated in the remote control unit. In one embodiment the dispensing patch unit can be composed of two parts, a reusable part that contains all electronic (e.g. processor, sensors) and at least a portion of driving mechanisms (e.g. motor, gears) and a disposable part that contains insulin reservoir and power supply. The glucose sensing means (e.g. glucometer) can also be integrated in the reusable part of the patch unit of the device.

The bolus selector can be implemented in the remote control unit of the insulin infusion device. The bolus selector can also be implemented in the reusable part of the dispensing patch unit of the device. The bolus selector can also be implemented in both the reusable part of the dispensing patch unit of the device and the remote control unit of the device.

In one implementation, the bolus selection method can be implemented in the dispensing patch unit that can continuously monitor body glucose levels and can concomitantly deliver insulin into the body. The dispensing patch unit can comprise a reusable part and a disposable part.

In one implementation, the insulin dispensing and glucose sensing capabilities can be combined into a semi-closed loop system, in which a processor-controller apparatus can regulate the dispensing of basal insulin according to the sensed glucose concentration and the meal boluses can be controlled by the bolus selector. Other implementations of the semi-closed loop systems are possible.

The bolus selector can be implemented in the remote control unit of the device. The bolus selector can also be implemented in the reusable part of dispensing patch unit of the device, or in both the reusable part of the dispensing patch unit of the device and the remote control unit of the device.

In one implementation, the bolus selection method can be implemented in a sensing means for sensing blood glucose, e.g. glucometer, continuous glucose monitor (“CGM”), or a means for continuously sensing subcutaneous interstitial fluid glucose or for any other glucose sensing means (e.g. non invasive glucose sensors, iontopheresis based sensors, etc.). In some embodiments, the sensing means can be a “stand alone” device or be a part of a system for insulin delivery.

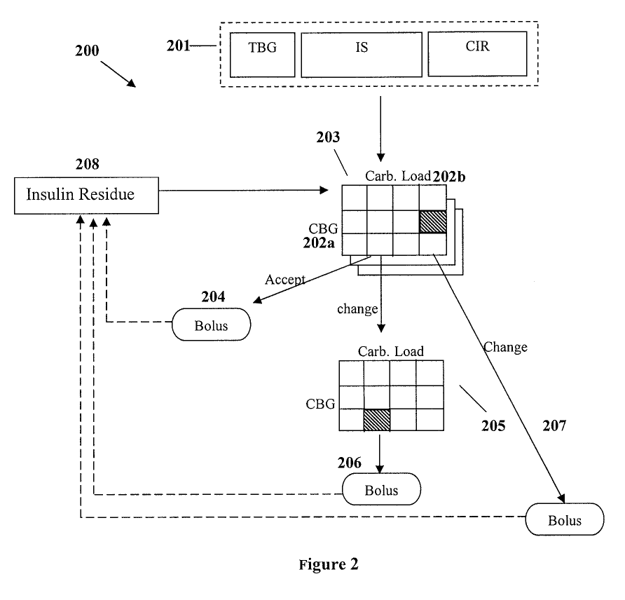

With continued reference to FIG. 1, FIG. 2 illustrates a block diagram representing one implementation of the method (200) for selecting a recommended insulin bolus dosage by the bolus selector (2000). In one implementation, a grid of insulin bolus dosages is retrieved from the memory of the bolus selector (2000) for selection of the user's specific bolus dose.

In one example, stored bolus-grids contain bolus doses that have been pre-determined for each combination of carb content and blood glucose content. The user may select the bolus dose from the retrieved bolus-grid, which corresponds to the combination of contemplated carb intake and results of the actual measurements of the blood glucose level. In another example, the bolus doses can be pre-determined using the following parameters: IS, CIR, target BG and residual insulin.

In one implementation, the patient's specific bolus-grid can be determined by IS, CIR and target BG values (201) that can be loaded by the patient, and insulin residue value (208) that can be automatically loaded according to the previous administered bolus. In one implementation, insulin residue values can be displayed periodically or at patient discretion.

The recommended dose from the displayed bolus-grid can be selected at 203. The selection can be based on the contemplated carb intake and blood glucose (BG) level. These parameters can be presented within the bolus-grid. In this example, rows represent carb ranges (202b), and columns represent current BG ranges (202a).

Blood glucose levels (202a) can be obtained from any suitable glucose sensor, such as a glucometer and continuous subcutaneous glucose sensor. In one implementation, the contemplated carb load (202b) can be evaluated by the user.

Residual insulin value (208) can be obtained from previous bolus dose and elapsed time, for example, as shown in Table 4. The input of the residual insulin (shown as dashed lines) allows retrieval of the stored bolus-grid with bolus dosages pre-determined according to values of residual insulin.

For example, according to Table 4, if 2 units of insulin were administered one hour prior to the current bolus, 1.6 units of insulin are not absorbed and reside in the body (residual insulin=1.6). In this case, the retrieved bolus-grid has corresponding pre-determined boluses that are lower by 1.6 units than the pre-determined boluses with no residual insulin (residual insulin=0).

In one implementation, the user can accept the selected bolus and bolus delivery (204). In another implementation, the user can manually modify the selected bolus by navigation along the bolus-grid (205) and consequently select an alternative bolus (206). In that implementation, the user can accept or reject the selected dose, or choose an alternative dose (not from the bolus-grid) (207).

Whether the bolus is selected by the bolus selector (203), modified and selected (205), or chosen (207), bolus delivery can be activated by the remote control unit (1008) or by manual bolus buttons located on the dispensing patch unit (1010). In one embodiment, other infusion devices for insulin delivery can also be used (e.g. insulin pump, remote-controlled insulin pump, dispensing patch unit, injection pen, insulin jet injector, transdermal insulin delivery device, subcutaneous insulin delivery device, implantable insulin delivery device, etc.).

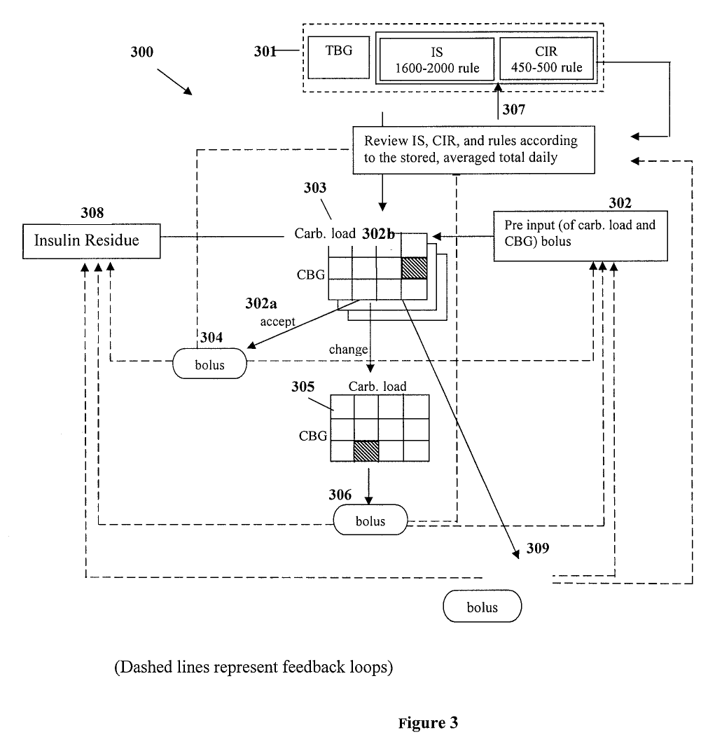

FIG. 3 shows a block diagram of another embodiment of the bolus selector method (300) for selecting a recommended insulin bolus dose. At 301, the bolus selector can be setup according to the user's individual parameters: carbohydrate to insulin ratio (CIR) and insulin sensitivity (IS). These parameters can be obtained, for example, according to the “450-500 rules” and “1600 to 2200 rules” respectively (Tables 2 and 3).

In one implementation, the user can also preset the target blood glucose (TBG) levels. TBG can vary throughout the day and can also be adjusted to allow pre-meal appropriate bolus-grid retrieval.

In one implementation, the CIR and IS values can repeatedly be adjusted according to last days stored bolus data (307). For example, the CIR and IS values can be selected from Tables 2 and 3 respectively by choosing the appropriate rule and the last days average insulin total daily dose (TDD). In some implementations, if the 500 and 1800 rules are selected and the average TDD is 501 U/day, the CIR and IS are 10 gram/unit and 36 mg/dl/unit respectively. After adjustment of the CIR and IS value, appropriate bolus-grids that correspond with the revised IS and CIR parameters can be retrieved by the bolus selector.

In one implementation, the rules applied can also be repeatedly adjusted according to the last days stored bolus data. The optimal rules to be applied can be determined by the percentage of the basal dose from the total daily dose (TDD). For example, if the basal dose is 50% of the TDD, the optimal applied rules should be the “500 rule” and “2000 rule” for the CIR and IS respectively. If the basal dose is 40% of the TDD, the applied rules should be the “450 rule” and “1800 rule” for the CIR and IS respectively. If the basal dose is 60% of the TDD, the applied rules should be the “550 rule” and “2200 rule” for the CIR and IS respectively.

The average bolus dose delivered during selected time interval in the last few days can optionally be presented to the user of the bolus selector at (302). For example, the day can be divided into five time intervals (e.g. 6:00-10:00, 10:00-14:00, 14:00-18:00. 18:00-22:00, 22:00-6:00) and the average of the total amount of insulin delivered during the corresponding time interval in the last several days would be the presented value (302). This value could be displayed as a stand alone parameter or as a marked cell within the bolus grid and can be used as an adjunct recommendation for bolus selection. In some implementations, the time interval can also be selected from a certain basal profile (e.g. only the boluses given between 6 to 10 am in the last 7 “weekend” profiles are averaged together).

The data regarding the time and the dosage of the previous boluses can be stored in the memory (2020), as shown in FIG. 1, and can be displayed periodically or based on the patient's discretion.

The bolus dose can be selected from the retrieved bolus-grid at (303). In this example, rows represent carb ranges (302b), and columns represent BG range (302a). In some implementations, blood glucose levels (302a) can be obtained from any suitable glucose sensor such as a glucometer, continuous subcutaneous glucose sensor etc. The carb load (302b) can also be evaluated by the user.

The appropriate bolus-grid with pre-determined bolus doses according to insulin residue value (e.g. as shown in Table 4) can be retrieved at (308). The user may accept or reject the selected dose, or choose an alternative dose (not from the bolus grid) (309). Whether the bolus is selected by the bolus selector (304), modified (305) and selected (306), recommended according to history (302) or chosen (309), bolus delivery can be activated by the remote control unit (1008) or by manual bolus buttons located on the dispensing patch unit (1010).

In one implementation, the selected bolus dose can be delivered automatically. In that case, the patient can be notified by tactile, audible or vibrational alerts. The patient can also have an option to suspend the automatic delivery. In one implementation, the dispensing device can be configured as a skin secured pump. In another embodiment, other devices for insulin delivery can also be used (e.g. insulin pumps, injection pens, etc.)

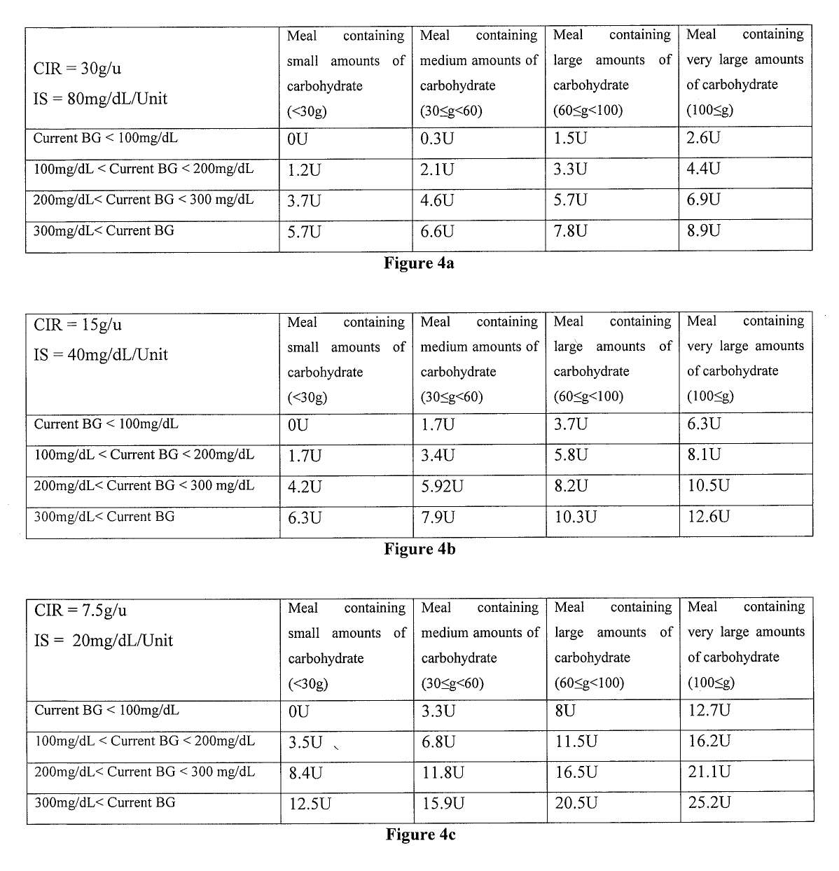

FIGS. 4(a)-4(c) show exemplary bolus-grids with pre-determined insulin bolus dosages corresponding to carb content and current blood glucose measurements. FIG. 4(a) shows a bolus-grid which can be suitable for a patient with a high carbohydrate-to-insulin ratio (CIR) (e.g. 30 g/unit) and high insulin sensitivity (IS) (e.g. 80 mg/dL/unit). FIG. 4(b) shows a bolus-grid which can be suitable for a patient with an average carbohydrate-to-insulin ratio (e.g. 15 g/unit) and average insulin sensitivity (e.g. 40 mg/dL/unit). FIG. 4(c) shows a bolus-grid which can be suitable for a patient with a low carbohydrate-to-insulin ratio (e.g. 7.5 g/unit) and low insulin sensitivity (e.g. 20 mg/dL/unit).

FIGS. 5-10 show various embodiments of interface configuration that can be displayed (2040) by the bolus selector (2000). For example, FIG. 5 shows one implementation of a user's interface for the bolus selector (2000). In this implementation, the user can select the required dose by scrolling the buttons (60, 61) and navigating between different numerical ranges quantitatively representing the blood glucose and the carb load of the intake.

The bolus selector (2000) can allow the user to select the bolus value in the bolus-grid that corresponds to a particular combination of carb intake and glucose level ranges. In another implementation, one marked (e.g., underlines, highlighted, blinking, color coded, etc.) cell within the selected bolus grid (64) can show the average dose delivered in previous boluses (e.g. average of boluses delivered in the last week during the same time interval). The user can either accept this recommended dose, navigate along the grids and select a different dose (as mentioned above), or reject the bolus selection process.

According to another embodiment (not shown), the average bolus dose of the doses delivered in previous boluses can be presented to the user as a stand alone parameter, and not as a value within a grid as depicted in the figure.

FIG. 6 shows another implementation of a user's interface for the bolus selector (2000). In that implementation, two couples of navigation buttons (60, 61) can be used to selectively navigate between different options qualitatively representing the current blood glucose and the carb load of the intake.

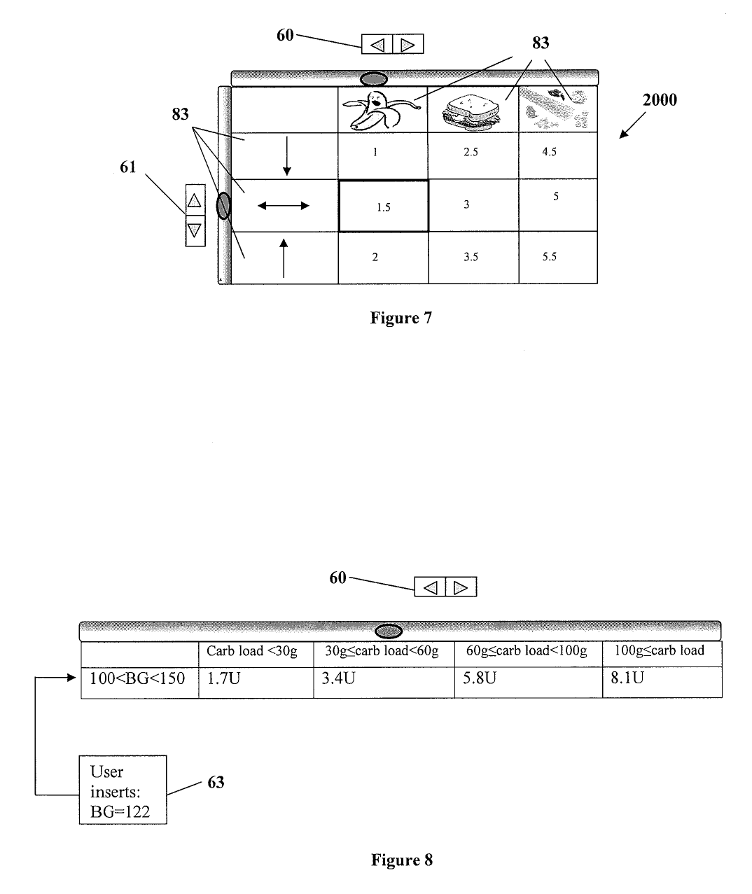

FIG. 7 shows another optional user's interface for the bolus selector (2000). The user can use two couples of navigation buttons (60, 61) to selectively navigate between different options (83) representing graphically as an animation the current blood glucose and the carb load of the intake. For example, young children and illiterate users may especially benefit from the graphical user's interface illustrated in FIG. 7.

FIG. 8 shows an additional embodiment of the user's interface for the bolus selector (2000), in which only a row of the bolus grid that corresponds to the relevant BG range is displayed. One couple of the navigation buttons (60) can be used to navigate between different numerical ranges representing the carb load of the intake. According to this embodiment, blood glucose value input (63) (e.g. BG=122) can be ascribed by the bolus selector (2000) to the relevant range of BG values (e.g. 100<BG<150).

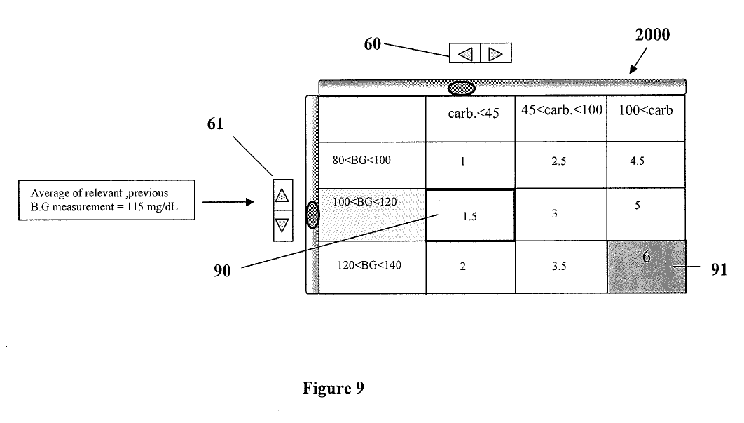

FIG. 9 shows an implementation of the user's interface for the bolus selector (2000), in which a suggestion for a selected dose is presented in the grid as a marked cell (90). The suggested bolus dose can correspond to the average levels of previous glucose measurements and carbs intakes at the relevant time interval. For example, in the last week during the time interval 12:00-14:00, the average glucose levels were 115 mg/dL and the average carbs intake was 30 gram. Accordingly, the bolus selector (2000) ascribes the average value (e.g. 115 mg/dL) to the relevant range of BG values (e.g. 100<BG<120) and the average carb intake (e.g. 30 gram) to the relevant range of carb value (<45 gram). The cell that corresponds to this combination (90) contains the suggested dose (i.e. 1.5 units).

The actual current BG and meal content and their corresponding grid cell and bolus dose can also be presented on the bolus grid of the bolus selector (2000). This dose could overlap with the average suggested dose (90) or not (91) depending on the difference between the average and current status. For example, the suggested dose is 1.5 units (90), however the user today between 12:00 and 14:00 is planning an unusually large meal and the BG is higher than normal. In this case the required corresponded bolus is 6 units (91).

Like in other implementations, two couples of navigation buttons (60) and (61) can be used to selectively navigate between different numerical ranges corresponding to the current blood glucose and the carb load of the intake.

In one implementation, the bolus selector (2000) may comprise many ranges of BG (e.g. resolution=20 mg/dL: 40-60 mg/dL, 60-80 mg/dL, 80-100 mg/dL, etc.). These BG ranges can be presented as columns in a grid with carbs ranges presented as rows. A BG range can also be presented as one row corresponding to the proper BG value (e.g. BG-122 mg/dL corresponds to the 120-140 mg/dL range). The user can scroll among carbs load ranges to select the bolus dose, from those displayed in the BG row.

In some implementations, if the BG value was not measured, the bolus selector (2000) can retrieve a bolus-grid, in which the bolus dose was pre-determined according to the average doses value +/−2 ranges on either side. The user can then scroll among carb ranges and BG ranges that are likely to comply with the user's glycemic state.

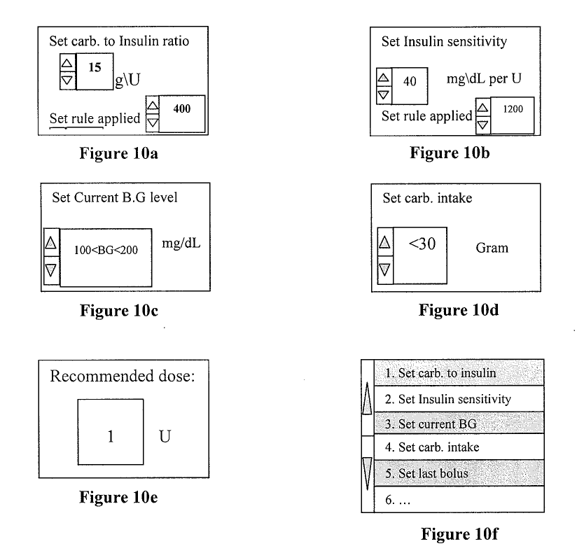

FIGS. 10a-10f show another example of a user interface that can be used by the bolus selector (2000). FIG. 10a shows an example of a window for loading carbohydrate-to-insulin ratio (CIR) and the corresponding rule. FIG. 10b shows an example of a window for loading insulin sensitivity (IS) and the corresponding rule. FIG. 10c shows an example of a window for loading current blood glucose levels. FIG. 10d shows an example of a window for loading carbs meal intake. FIG. 10e shows an example of a window, displaying a bolus dose. FIG. 10f shows an example of a main window of the bolus selector (2000) through which the user can select any of the windows presented in FIGS. 10a-e. Additional windows may be accessible via the main window (e.g. a window for downloading last bolus data).

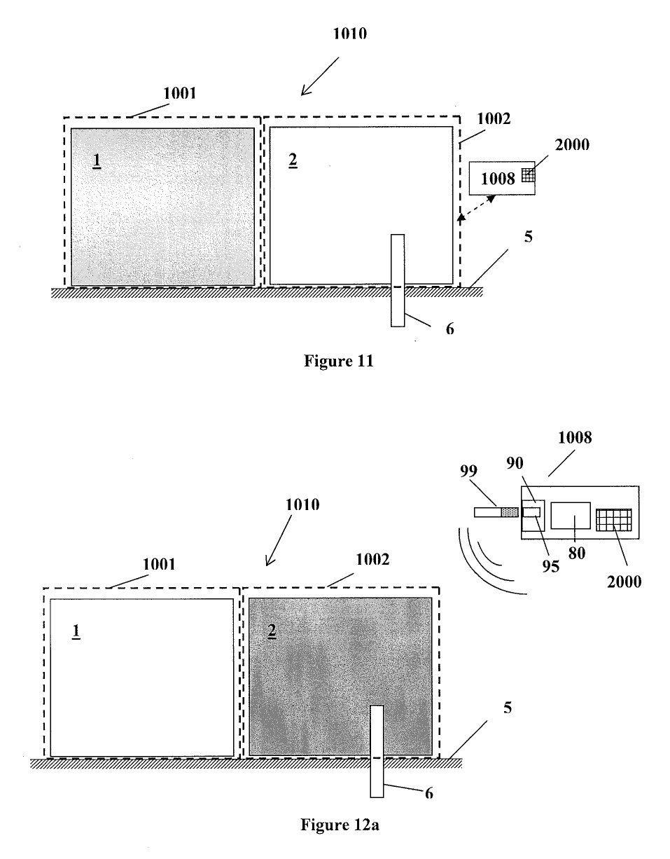

FIG. 11 shows one implementation of the insulin delivery device, wherein the dispensing patch unit (1010) is composed of two parts located in two housings (1001, 1002)—a reusable part (1) and a disposable part (2). For example, the relatively inexpensive components of the device can reside in the disposable part (2) (e.g. cannula (6)) and the relatively expensive components can reside in the reusable part (1). In another implementation (not shown), the cannula (6) can be attached to a skin adhered cradle unit allowing connection and disconnection of the dispensing patch unit (1010) from the needle unit, as disclosed in detail in our patent application U.S. Ser. No. 60/876,679 (herein incorporated by reference in its entirety). The device may also comprise a remote control unit (1008) with an integrated bolus selector (2000). Programming can be carry out by the remote control (1008) or by buttons located on the dispensing patch unit (1010).

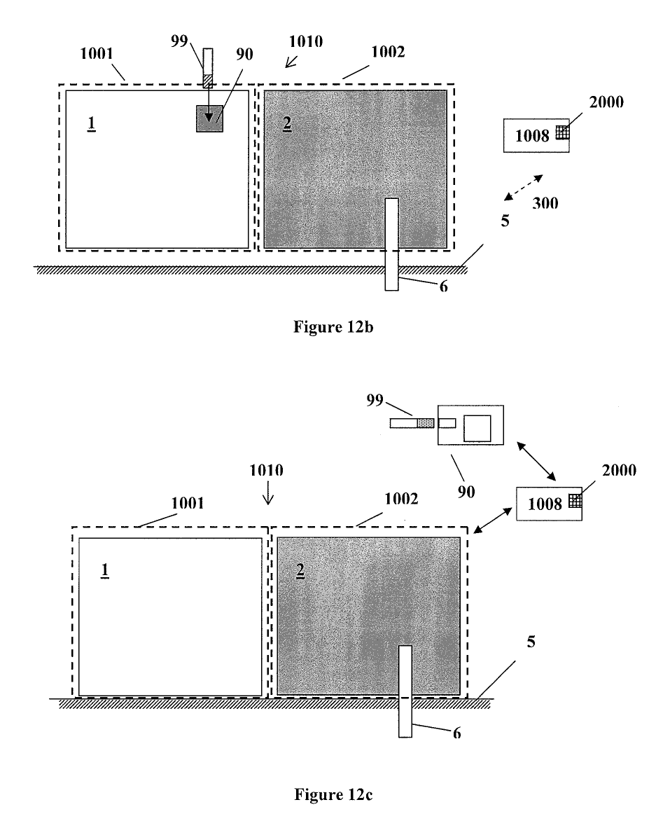

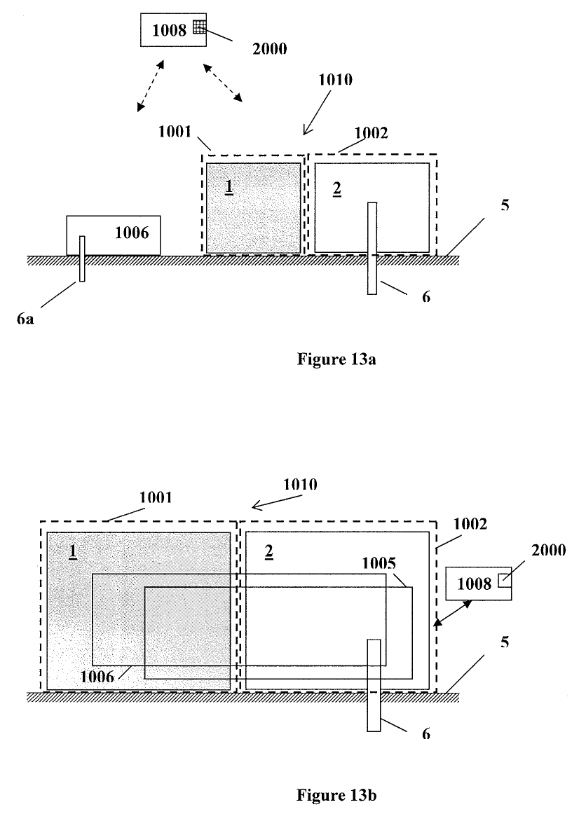

FIGS. 12a-12c show three different implementations of the device. Each of these implementations contains a glucometer (90) to be used as blood glucose (BG) inputs for the bolus selector (2000).

FIG. 12a shows a glucometer (90) located in the remote control unit (1008) of the device. The glucometer (90) can comprise an opening (95) for receiving of a test strip (99). For example, the user can extract blood from the body, place the blood on the test strip (99) and insert the strip into the opening (95). The glucose readings (90) can be displayed on screen (80) of the remote control unit (1008).

FIG. 12b shows a glucometer (90) located in the reusable part (1) of the dispensing patch unit (1010). A communication channel (300) between the glucometer (90) residing in the dispensing patch unit (1010) and the bolus selector (2000) residing in the remote control unit (1008) can be maintained, allowing programming, data handling, and user inputs.

FIG. 12c shows an embodiment in which glucose readings can be directly or remotely (90) received from an independent glucometer.

FIGS. 13a-13b illustrate some implementations, in which blood glucose readings can be manually loaded to the bolus selector (2000), or automatically received by the bolus selector (2000), from a continuous subcutaneous glucose monitor (1006). A communication channel between the continuous subcutaneous glucose monitor (1006) and the bolus selector (2000) residing in the remote control unit (1008) can be maintained, allowing programming, data handling, and user inputs.

FIG. 13a shows an embodiment in which the current blood glucose concentration can be received from the independent continuous subcutaneous glucose monitor (1006) provided with the probe (6a). The glucose concentration levels measured by the monitor (1006) can be loaded into the bolus selector (2000) by the patient.

FIG. 13b shows an embodiment in which the continuous subcutaneous glucose sensing means (1006) is located in the dispensing patch unit (1010) of the insulin delivery device.

As disclosed in our previous PCT application PCT/IL07/000,163, herein incorporated by reference in its entirety, the insulin dispensing apparatus (1005) and glucose sensing means (1006) constitute, in the illustrated embodiment, a single delivery device, and can use a single cannula (6) for both dispensing and sensing. Alternatively (not shown), the sensing means and the dispensing apparatus can have separate cannulae that penetrate the skin (5) and reside in the subcutaneous tissue. The delivery device of this embodiment may be composed of two parts—a reusable part (1) and a disposable part (2), each part has corresponding housing (1001, 1002).

In another implementation (not shown) the device can contain a closed loop or semi-closed loop system. Insulin can automatically be dispensed according to continuous glucose level monitoring (closed loop) or according to continuous monitoring and additional pre-meal bolus user inputs (semi-closed loop). The bolus selector (2000) can be used for bolus inputs in the semi-closed loop system.

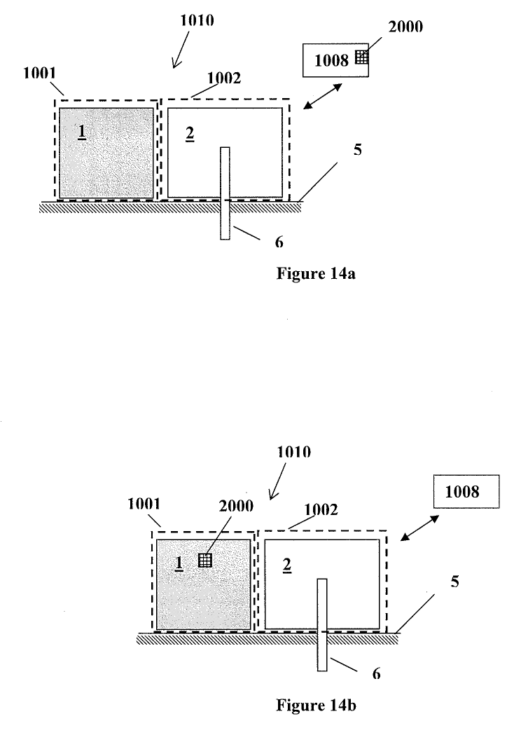

FIGS. 14a-14b show two implementations of the device with different location of a bolus selector (2000). In FIG. 14a, the bolus selector (2000) is located in the remote control unit (1008). In FIG. 14b the bolus selector (2000) is located in the reusable part (1) of the dispensing patch unit (1010).

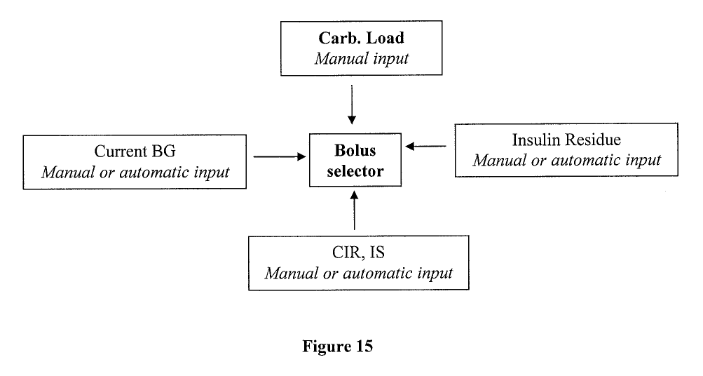

FIG. 15 shows the bolus selector data inputs that can be used for patient specific bolus-grid retrieval by the bolus selector (2000). For example, the carbs load and the current blood glucose levels can be acquired manually. In some implementations, the user can check his or her blood glucose level with the aid of a glucometer, a continuous subcutaneous glucose monitoring device, or any other suitable means known in the art for measuring blood glucose levels. In some implementations, the current blood glucose levels may be acquired automatically.

In one implementation, the device can comprise a glucometer and a communication channel between the bolus selector and the glucometer, to allow direct input of the measured blood glucose. In one embodiment, a communication channel can exit between the bolus selector and an independent glucometer, to allow direct input of the measured blood glucose.

In another embodiment, a continuous subcutaneous glucose monitoring apparatus can continuously transmit BG levels to the bolus selector. A communication channel may also exist between the bolus selector and an independent continuous subcutaneous glucose monitoring device, allowing direct transmission of the measured blood glucose.

The data concerning the residual insulin (time and dose of last bolus) may be obtained manually or automatically, allowing retrieval of the appropriate bolus-grid from the bolus selector and selecting the bolus dose.

The carbohydrate-to-insulin ratio (CIR) and insulin sensitivity (IS) can be used by the bolus selector according to the initial settings of the user. These values can be revised in accordance with the specific rules applied when setting the system (similarly to (307) in FIG. 3) and can be inputted manually or automatically.

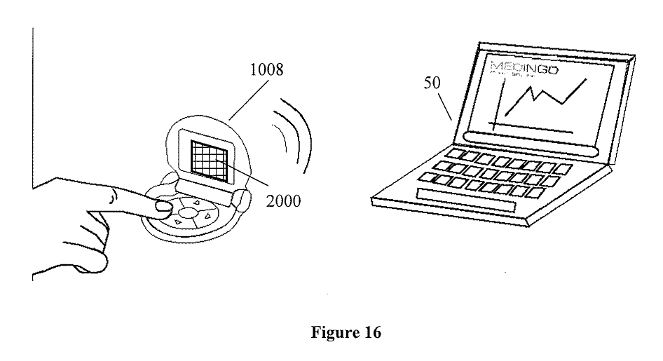

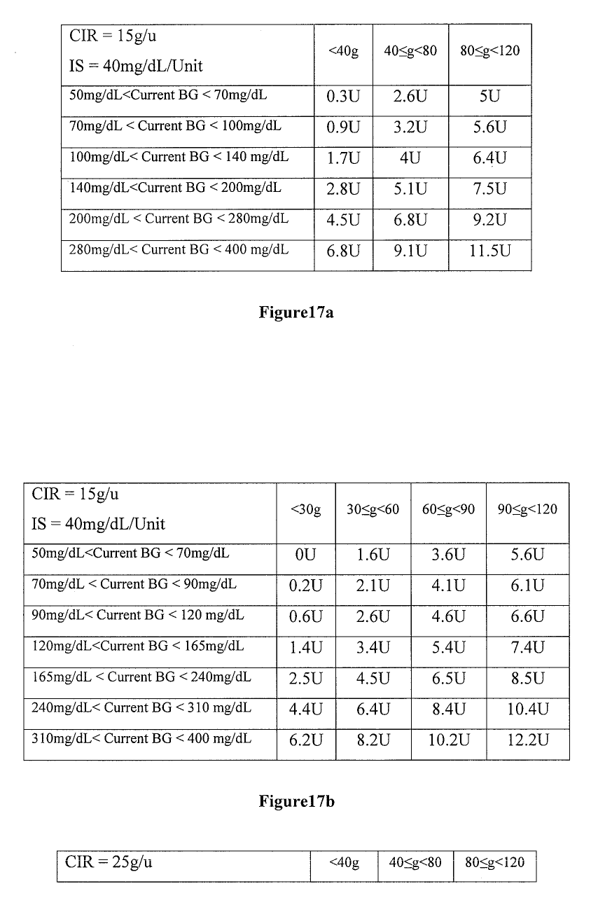

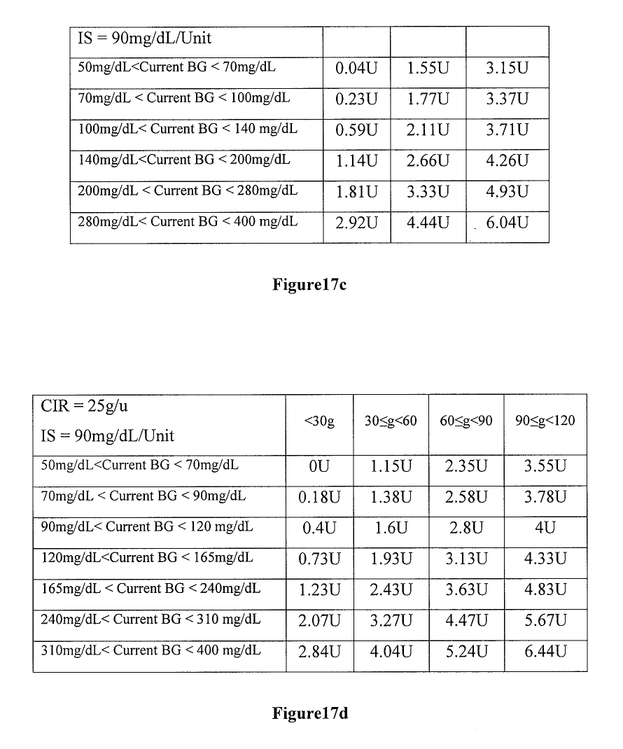

FIG. 16 illustrates another implementation of the device, where the bolus selector (2000), is located in a remote control unit (1008) and can communicate with an external PC (50). For example, in this implementation, only those bolus-grids that correspond to the user's particular preset IS value and CIR will be saved in the memory of the bolus selector (2000) in the remote control (1008) while the rest of the bolus-grids (i.e. bolus-grids that correspond to other IS values and CIR Ratios) will be saved in the memory of the external PC (50). If the diabetic state of the user changes, the bolus-grids that correspond to the new IS value and CIR may be downloaded from the external PC (50) to the bolus selector memory in addition to, or instead of, the previous stored bolus-grids.

In another implementation, the bolus-grids can be tailored to a specific user based on the user's setting of IS value and CIR, prior to the initiation of the system. For example, if the user's diabetic state changes, new bolus-grids, corresponding to the new IS value and CIR, can be pre-determined. In some implementations, the tailoring of the bolus-grids, prior to the system use, can be carried out directly via the remote control unit (1008). The bolus-grids can also be pre-determined with the aim of a PC (50) and downloaded afterwards to the bolus selector (2000) in the remote control unit (1008).

FIGS. 17a-17d provide examples of pre-determined bolus-grids that can be stored in the memory of the bolus selector and can be displayed following IS and CIR user specific values inputs.

FIG. 17a shows a bolus-grid comprising six ranges of blood glucose levels (vertical axis) and three ranges of carb load values (horizontal axis). The bolus-grid shows pre-determined bolus values referring to users with an IS value of 40 mg/dL/unit and a CIR of 15 g. FIG. 17b shows a bolus-grid comprising seven ranges of blood glucose levels (vertical axis) and four ranges of carb load values (horizontal axis). The bolus-grid shows pre-determined bolus values referring to users with an IS value of 40 mg/dL/unit and a CIR of 15 g. FIG. 17c shows a bolus-grid comprising six ranges of blood glucose levels (vertical axis) and three ranges of carb load values (horizontal axis). The bolus-grid shows pre-determined bolus values referring to users with an IS value of 90 mg/dL/unit and a CIR of 25 g. FIG. 17d shows a bolus-grid comprising seven ranges of blood glucose levels (vertical axis) and four ranges of carb load values (horizontal axis). The bolus-grid shows pre-determined bolus values referring to users with an IS value of 90 mg/dL/unit and a CIR of 25 g. In one implementation, the difference between the maximum BG value and the minimum BG value in a range can increase with increasing BG values.

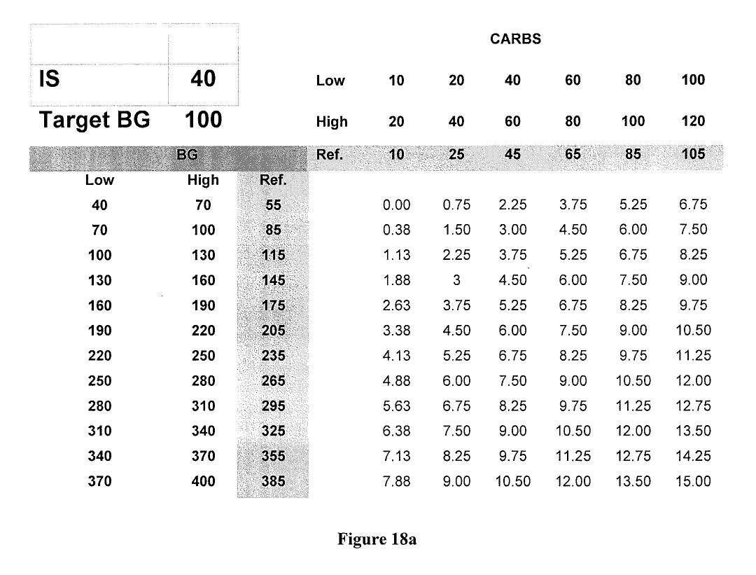

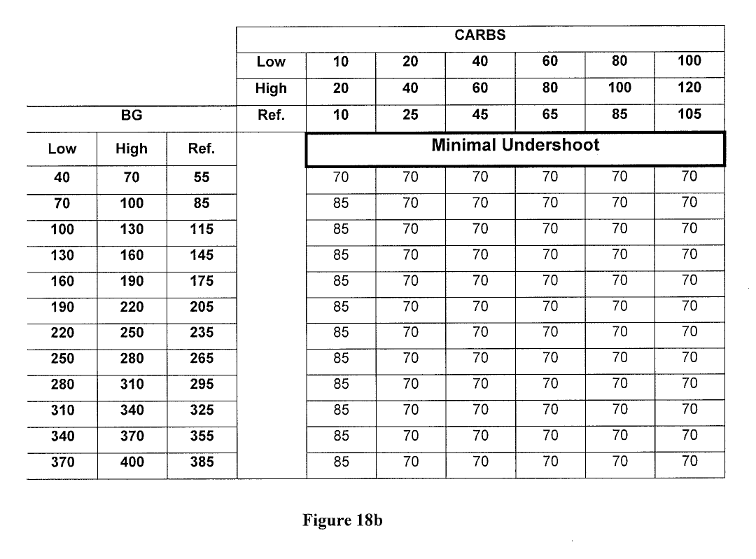

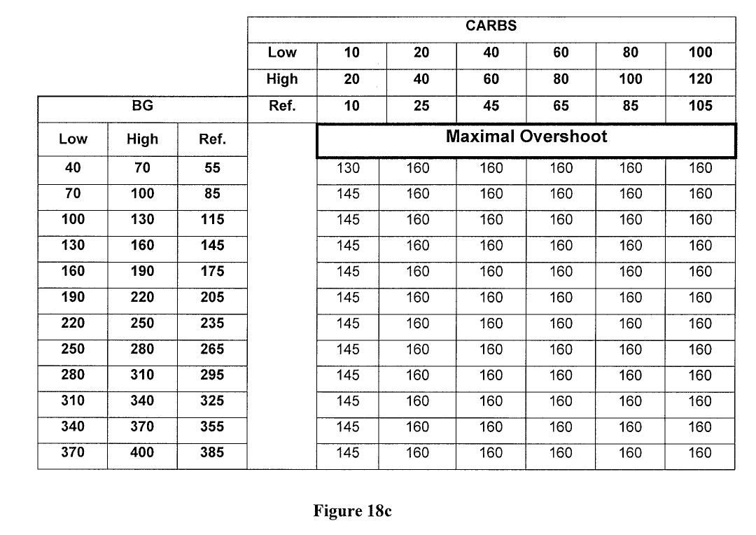

FIG. 18a shows another example of a pre-determined bolus-grid that may be stored in the memory of the bolus selector. The bolus-grid in the example can be suitable for a user with an IS value of 40 mg/dL/unit, an IS/CIR ratio of 3 (i.e. CIR=13.3 g/unit), and a target BG level of 100 mg/dL. As shown, each range (either of blood glucose (BG) levels or carbs) has a low boundary and a high boundary defining the range. Each range is represented by a discrete value designated as a reference value (“Ref.”) which is applied in the calculations of the pre-determined bolus.

In one implementation, the reference value is not necessarily the mid-range value but rather a value closer to the lower portion of each range. In some implementations, the reference value may be intentionally shifted during generation of the pre-determined bolus-grids, for example, higher than the mid-range value. In some examples, the reference value may be equal to the upper boundary value (e.g. in a range of 20 to 40, the reference value may be 40). The rationale for this shift is that it may minimize human error in estimating the carb load to be ingested. This error can be frequently caused by the tendency of users to underestimate the amount of carbohydrates.

In some implementations, during generation of the pre-determined bolus-grids, each cell value of a bolus-grid (presented in units of insulin) can be selected to lead the BG concentrations to fall within a pre-defined allowable clinical range of blood glucose levels. This clinical range can be bounded between two discrete values of blood glucose levels: a lower boundary referred-to as the “minimal undershoot”, and an upper boundary referred-to as the “maximal overshoot”. The minimal undershoot and the maximal overshoot can be, for example, 60 mg/dL and 200 mg/dL, respectively. If an insulin value within a cell causes the blood glucose level of the patient to be outside of the allowable range, (i.e. <60 mg/dL or >200 mg/dL), the patient can likely suffer from hypoglycemia or hyperglycemia, which may be hazardous.

According to some embodiments, the selection of a value of insulin bolus dose for a particular cell can be done using a verification process that tests the boundary values of the blood glucose range and the carbohydrate intake range corresponding to that cell.

For example, the cell value which corresponds to the blood glucose (BG) range 130 mg/dL to 160 mg/dL, and carbohydrate intake range of 40 grams to 60 grams can first be calculated according to the reference values, i.e. BG of 145 mg/dL and carbohydrate intake of 45 grams yielding the value of 4.5 units of insulin to be administered. The verification process can calculate a lower value based on the low boundary of the BG and carbohydrate intake ranges (i.e. BG=130 mg/dL and carbs=40 grams), and an upper value based on the high boundary of the ranges (i.e. BG=160 mg/dL and carbs=60 grams), resulting in a lower value of 3.76 Units and the upper value of 6 Units. The absolute difference between the lower value (3.76 Units) and the cell value (4.5 Units) equals to 0.74 Units, and is referred-to as “low_diff”. The low_diff difference can bring the BG level of the patient to approximately 70 mg/dL. For example, this can be calculated by using the following formula: TBG-low_diff*IS. Following the example presented above, the low_diff value can bring the BG level of the patient to 100-0.74*40=70.4 mg/dL.

A similar procedure can be done with the upper value: the absolute difference between the upper value (6 Units) and the cell value (4.5 Units) is determined to be 1.5 Units, and referred-to as “upper_diff”. This difference could bring the BG level of the patient to a value of approximately 160 mg/dL. If one of the values (70 mg/dL or 160 mg/dL) would have fallen outside of the clinical range defined by the minimal undershoot and the maximal overshot, then, in some implementations, the reference value and/or values of the BG and carbohydrate intake ranges can be re-assessed. The verification procedure described above can be carried out iteratively to determine the optimal cell value ensuring that the BG level of the patient remains in the allowed clinical range. In some embodiments, a single clinical range (i.e. minimal undershoot and maximal overshoot) can be defined for all the cells of the bolus-grid. In further embodiments, a different clinical range can be defined for each cell of the bolus-grid. In some embodiments, the patient/user can define/program parameters such as minimal undershoot, maximal overshoot and/or boundaries of the ranges, adjusting the bolus-grid to be tailored to his/her own individual needs. In one implementation, the BG levels corresponding to “low_diff” and “upper_diff” values can be pre-calculated and stored in a database table. For example, FIGS. 18b and 18c show the corresponding minimal undershoot and maximal overshoot tables, respectively. Specifically, the table 18b provides a list of minimal undershoot values for each of the BG ranges and for each of the six carbohydrate intake ranges presented in FIG. 18a. In one implementation, the tables 18b and 18c are not presented to the user.

For example, according to the table 18b, the minimal allowable undershoot value is 70 mg/dL. According to table 18c, the maximum allowable overshoot value is 160 mg/dL. In some embodiments, the undershoot and/or overshoot values, lower and/or upper boundaries of the ranges and other bolus-grid parameters can be adjusted.

Various implementations of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

Although a few variations have been described in detail above, other modifications are possible. For example, the logic flow depicted in the accompanying figures and described herein does not require the particular order shown, or sequential order, to achieve desirable results. Other implementations may be within the scope of the following claims.

What is claimed is:

1. A method for selecting a bolus dose of a drug in a drug delivery device comprising selecting the bolus dose from a pre-determined schedule of bolus doses, wherein each dose corresponds to a range of a body analyte levels.

2. The method according to claim 1, wherein the pre-determined schedule of doses corresponds to a bolus-grid.

3. The method according to claim 1, wherein the drug comprises insulin and wherein the range of the body analyte levels corresponds to a range of blood glucose levels.

4. The method according to claims 3, wherein the range of the blood glucose levels is represented as a qualitative descriptive parameter (QDP).

5. The method according to claim 4, wherein the QDP is expressed as a plurality of terms, each associated with a pre-determined blood glucose level or a pre-determined range of blood glucose levels.

6. The method according to claim 5, wherein the terms are selected from the group consisting of high, normal and low.

7. The method according to claim 3, wherein the bolus doses are determined as a function of a plurality of parameters.

8. The method according to claim 7, wherein the parameters are selected from the group consisting of time elapsed from a last meal, an amount of one or more previous bolus doses, target blood glucose (TBG) level, carbohydrate-to-insulin ratio (CIR) and insulin sensitivity (IS).

9. The method according to claim 7, wherein the parameters are selected from the group consisting of intensity of physical activity, duration of physical activity, menstrual cycle, concomitant administration of other drugs, the drug injection site, glycemic index, stress and fever.

10. A method for selection of a bolus dose of a drug in a drug delivery device comprising selecting the bolus dose from a pre-determined schedule of bolus doses, wherein each dose corresponds to a range of a nutritional consumable.

11. The method according to claim 10, wherein the pre-determined schedule of bolus doses corresponds to a bolus-grid.

12. The method according to claim 10, wherein the drug is insulin and wherein the nutritional consumable comprises at least one of carbohydrate, fat and protein.

13. The method according to claim 10, wherein the range of the nutritional consumable is represented as a qualitative descriptive parameter (QDP).

14. The method according to claim 13, wherein the QDP is expressed using a plurality of terms, each associated with a pre-determined range of the nutritional consumable.

15. The method according to claim 14, wherein the terms are selected from the group consisting of small, medium and large.

16. The method according to claim 10, wherein the bolus doses are determined as a function of a plurality of parameters.

17. The method according to claim 16, wherein the parameters are selected from the group consisting of time elapsed from a last meal, an amount of one or more previous bolus doses, target blood glucose (TBG) level, carbohydrate-to-insulin ratio (CIR) and insulin sensitivity (IS).

18. A method for selection of a bolus dose of a drug in a drug delivery device comprising selecting the bolus dose from a pre-determined schedule of bolus doses, wherein each bolus dose corresponds to one or more glucose levels and one or more nutritional consumables.

19. The method according to claim 18, wherein each bolus dose corresponds to a range of glucose levels and a range of nutritional consumables.

20. The method according to claim 18, wherein the pre-determined schedule of bolus doses corresponds to a bolus-grid.

21. An apparatus for selecting a bolus dose comprising:
means for providing a pre-determined schedule of bolus doses, wherein each bolus dose corresponds to a range of body analyte levels; and
means for selecting a bolus dose from the pre-determined schedule of bolus doses.

22. The apparatus according to claim 21, further comprising a remote control unit for controlling the means for selecting the bolus dose.

23. The apparatus according to claim 21, wherein the means for providing the pre-determined schedule of bolus doses comprise a display for displaying the pre-determined schedule of bolus doses.

24. The apparatus according to claim 23, wherein the means for selecting the bolus dose comprise either a touch sensitive screen which operates in conjunction with the display or one or more selection buttons/switches.

25. The apparatus according to claim 21, further comprising a dispensing unit for dispensing bolus doses into a user.

26. The apparatus according to claim 21, further comprising at least one body analyte level monitor for monitoring a body analyte level of a user; and, a processor for matching the body analyte level of the user to the range of body analyte levels.

27. An apparatus for selecting an insulin bolus dose comprising:
means for providing a pre-determined schedule of bolus doses as a function of one or more inputs, said inputs comprising at least one of a carbohydrate-to-insulin ratio (CIR), insulin sensitivity (IS) value and a body analyte level of a user;
means for adjusting the pre-determined schedule of bolus doses based on the inputs; means for selecting a bolus dose from the pre-determined adjusted schedule of bolus doses.

28. The apparatus according to claim 27, wherein the means for providing the pre-determined schedule of bolus doses comprise a display for displaying the pre-determined schedule of bolus doses.

29. The apparatus according to claim 27, wherein the means for providing the pre-determined schedule of bolus doses further includes means for indicating a bolus dose based on averaged values of previously used blood glucose levels and a carbohydrate intakes during a time interval.

30. The apparatus according to claim 27, wherein the means for providing the pre-determined schedule of bolus doses further includes means for presenting a subset of the predetermined schedule of bolus doses which corresponds to an averaged value of previously used blood glucose levels during a time interval.

31. The apparatus according to claim 28, wherein the means for selecting the bolus dose comprise either a touch sensitive screen which operates in conjunction with the display or one or more selection buttons/switches.

32. The apparatus according to claim 27, further comprising a dispensing unit for dispensing bolus doses into the user.

33. The apparatus according to claim 27, further comprising at least one monitor for monitoring the body analyte level of the user.

34. The apparatus according to claim 27, wherein the means for adjusting the schedule of bolus doses comprises a selecting means for selecting at least one of the CIR and IS values from a schedule.

35. The apparatus according to claim 27, wherein the means for adjusting the schedule of bolus doses comprises means for selecting rules for determining the CIR and IS values.

36. The apparatus according to claim 27, further comprising a display configured to display values corresponding to the inputs in a form of a list, a table or a graphical indication.

37. A method for selecting an insulin bolus dose comprising:
receiving at least one value selected from the group consisting of a carbohydrate-to-insulin ratio (CIR), an insulin sensitivity (IS) value and a target blood glucose (TBG) level of a user;
retrieving a pre-determined schedule of bolus doses based on the at least one received value;
presenting the pre-determined schedule of bolus doses for selection to the user;
selecting at least one bolus dose from the pre-determined schedule of bolus doses based on the user's blood glucose level and the user's nutritional consumable load.

38. The method according to claim 37, wherein the carbohydrate-to-insulin ratio (CIR) and the insulin sensitivity (IS) values are selected from a schedule by the user.

39. The method according to claim 37, wherein the schedule is determined by using rules for estimation of the carbohydrate-to-insulin ratio (CIR) and the insulin sensitivity (IS) values.

40. The method according to claim 39, wherein the estimation of the carbohydrate-to-insulin ratio (CIR) and the insulin sensitivity (IS) values is based on averaged values of total daily doses (TDD).

41. A system for drug dispensing comprising:
a remote control unit with a display for providing a pre-determined schedule of bolus doses, wherein each dose corresponds to a range of nutritional consumable load and to a range of body analyte levels; a user interface for selecting a bolus dose from the pre-determined schedule of bolus doses; a dispensing unit for dispensing the bolus dose into a user, wherein the dispensing unit receives instructions for dispensing the bolus dose from the remote control unit.

42. The system of claim 41, wherein the dispensing unit comprises:
a reusable part which includes a processor and a driving mechanism; and,
a disposable part which includes a reservoir.

43. The system of claim 41, further comprising a cradle unit.

44. The system of claim 41, further comprising:
a dispensing unit for delivering fluid into the user's body;
a sensing means for sensing, measuring and monitoring an analyte concentration level in the user's body.

45. The system of claim 44, wherein the fluid is insulin and the analyte is glucose.

46. The system of claim 44, wherein the dispensing unit and the sensing means operate as a semi-closed loop system.

47. A method for delivery of an insulin bolus dose to a user comprising:
selecting at least one bolus dose from a pre-determined schedule of bolus doses based on the user's blood glucose level and the user's nutritional consumable load, delivering the at least one selected bolus dose to the user.

48. The method of claim 47, in which the delivering of the at least one selected bolus dose is carried out by a delivery device selected from the group consisting of: insulin pump, insulin pump with a remote control unit, dispensing patch unit, injection pen, insulin jet injector, transdermal insulin delivery device, subcutaneous insulin delivery device, implantable insulin delivery device.

49. The method of claim 48, in which the delivering of the at least one selected bolus dose is carried out upon activating the remote control unit.

50. The method of claim 48, in which the delivering of the at least one selected bolus dose is carried out upon activating bolus buttons located at the dispensing patch unit.
